(19) United States
(12) Patent Application Publication
(10) Pub. No.: US 2009/0048335 A1
(43) Pub. Date: Feb. 19, 2009

(54) PROCESS FOR PREPARING SIMVASTATIN

(76) Inventors: Venkata Naga Mandava, Hyderabad (IN); Radha Krishna Singamsetty, Hyderabad (IN); Satish Kumar Vujjni, Hyderabad (IN); Vadali Ravi Kumar, Hyderabad (IN); Thambidurai Marappa Gounder, Erode (IN); Dipal Ranjan Bhowmik, Midnapore (IN); S. Venkatesh, Hyderabad (IN); Kedar K. Nerurkar, Mumbai (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/192,256

(22) Filed: Aug. 15, 2008

Related U.S. Application Data
(60) Provisional application No. 61/038,906, filed on Mar. 24, 2008.

(30) Foreign Application Priority Data
Aug. 17, 2007 (IN) 1836/CHE/2007

(51) Int. Cl. A61K 31/351; C07D 309/30

(52) U.S. Cl. 514/460; 549/292

(57) ABSTRACT

Preparation of simvastatin.

### INTRODUCTION

The present invention relates to processes for the preparation of 2,2-dimethylbutanoic acid (1S,3R,7S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester, also named butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1 S-[1α,3α,7β,8β(2S*,4S*),8aβ]], hereinafter referred to by the adopted name “simvastatin,” which has structural Formula I.

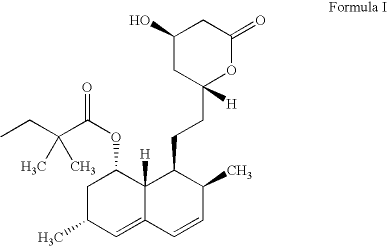

Simvastatin is a potent anti-hypercholesterolemic agent. It inhibits the enzyme 3-hydroxy-3-methyl-glutaryl coenzyme A reductase (“HMG-CoA reductase”), which catalyzes the formation of mevalonic acid, and thus inhibits cholesterol biosynthesis. It also increases the number of cellular LDL-receptors that remove the LDL cholesterol circulating in the blood, and thereby lower blood cholesterol levels. Pharmaceutical products containing the compound are commercially available using the trademark ZOCOR.

Several synthetic methods are reported in the literature for the preparation of simvastatin. In most of the synthetic methods known to manufacture simvastatin of Formula I, a compound of the Formula II(a) appears to be the most common intermediate, which is generically called the ammonium salt of simvastatin. The ammonium salt of simvastatin is lactonized to produce simvastatin, which is an essential step of the synthesis. Hence, it is desired to have an efficient method for lactonization that can produce simvastatin with consistent yield and quality.

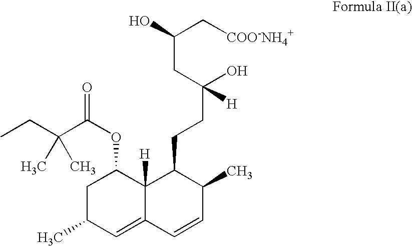

U.S. Pat. No. 4,916,239 discloses a process for the lacotnisation comprising treating a dihydroxy acid ammonium salt with a mixture of a water miscible organic solvent, water and an acid catalyst under a inert gas atmosphere for about 2-3 hours at 20-25° C., followed by precipitating the product by addition of water to produce the lactone as a crystalline mass.

U.S. Pat. No. 5,917,058 discloses a process for the preparation of simvastatin comprising treating the open ring hydroxy acid form of simvastatin with an excess of acetic acid in the absence of strong acid catalyst under mild heating condition at about 55° C. and adding an antisolvent to recrystallize a lactone form from the reaction mixture.

U.S. Pat. No. 5,939,564 discloses a process for the preparation of simvastatin using an organic base with an organic or inorganic acid such as pyridine hydrobromide, pyridine hydrochloride, or pyridinium, p-toluene sufonate. The organic solvent comprises a lower alkanol, non-alcoholic polar solvent or a non-alcoholic polar solvent for lactonization.

U.S. Pat. No. 6,380,401 discloses a one-pot process, involving treating dihydroxy acid with a strong mineral acid in a cold, aprotic, water miscible solvent to affect lacotnisation followed by addition of excess acid to effect crystallization of the lactonized product from the reaction mixture.

The above mentioned processes suffer from several disadvantages such as use of high boiling point solvents or mixtures of solvents, which need to be removed completely by distillation for isolation of the product from the reaction mixture. Some of the processes require anhydrous conditions, or they are lengthy or require repetitive and excessive amounts of strong mineral acids, or they involve tedious work ups.

Thus, there is a need in the art to provide a simple, cost effective, efficient and industrially feasible process for the synthesis of simvastatin of Formula I, which process is more convenient and more efficient than the previously known methods.

### SUMMARY

In an aspect, the present invention provides improved processes for preparing simvastatin of Formula I, by lacotnisation of the corresponding 3,5-dihydroxy acid or salt of Formula II,

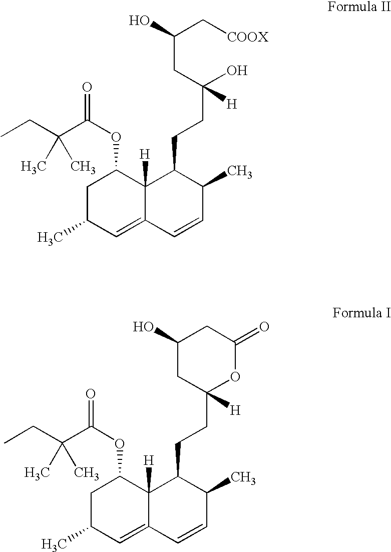

where X is H, Na, K, or NH₄, an embodiment comprising:

a) providing a solution of a 3,5-dihydorxy acid or salt of Formula II in a solvent;

b) adding an acid in a single lot, in an amount equal or greater than the amount sufficient to protonate the acid or salt; and

c) adjusting the pH of the solution of step (b) from about 5 to about 7.

In an aspect the invention includes processes for preparing solid premixes comprising simvastatin with a uniform particle size distribution, an embodiment comprising grinding simvastatin with an antioxidant solution.

### DETAILED DESCRIPTION

The present application relates to improved processes for the preparation of simvastatin and premixes containing simvastatin.

In an aspect, the present application provides improved processes for preparing simvastatin of Formula I, by lacotnisation of a 3,5-dihydroxy acid or salt of Formula II,

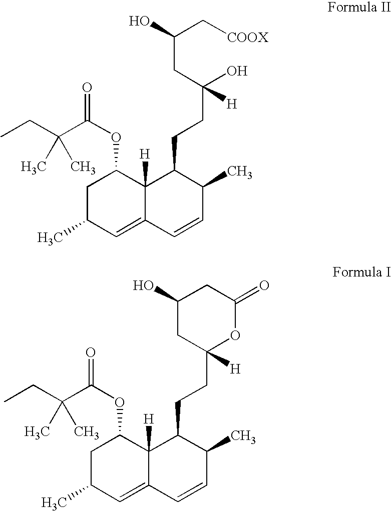

where X is H, Na, K, NH₄, an embodiment comprising:

a) providing a solution of a 3,5-dihydorxy acid or salt of Formula II in a solvent;

b) adding an acid in a single lot, in an amount equal or greater than the amount sufficient to protonate the acid or salt; and

c) adjusting the pH of the solution of step (b) from about 5 to about 7.

Step a) involves providing a solution of a 3,5-dihydorxy acid or salt of Formula II in a solvent.

The solution of the 3,5-dihydroxy acid or salt may be obtained by dissolving the 3,5-dihydroxy acid or salt in a suitable solvent, or a solution may be obtained directly from a reaction in which the 3,5-dihydroxy acid or salt is formed.

Solvents that are useful to provide the solution include any water miscible solvent without limitation, such as: nitrile solvents including acetonitrile, propionitrile and the like; alcohols such as methanol, ethanol, isopropyl alcohol, n-butanol, and the like; ketones such as acetone, ethyl methyl ketone, methyl isobutyl ketone and the like; and mixtures thereof.

The amount of solvent that is used to dissolve the 3,5-dihydroxy acid or salt can range from about 1 to about 10 times the weight of the 3,5-dihydroxy acid or salt. The dissolution temperatures can range from about 20 to 100° C., depending on the solvent used for dissolution. Any other temperatures are also acceptable as long as the stability of the 3,5-dihydroxy acid or salt is not compromised and a clear solution is obtained.

Step b) involves adding an acid to the solution in a single lot, in an amount equal or greater than a sufficient amount to protonate the acid or salt.

Suitable acids useful to protonate the acid or salt include organic and inorganic acids. Suitable inorganic acids include, without limitation, sulphuric acid, hydrochloric acid and the like, and suitable organic acids include but are not limited to acetic acid, formic acid and the like. Combinations of acids can be used, and mixtures of one or more acids in various proportions with water.

The molar ratios of acid to 3,5-dihydroxy acid or salt used in this reaction step range from about 2 to about 10, or about 4 to about 8, or about 5 to about 7, or about 6.5, and the suitable pH to protonate is less than 7, or less than about 5, or about 1.

The amounts of water used to dilute the acid in this reaction step range from about 1 to about 10 times, or about 1 to about 5 times, or about 2 times, the volume of the acid.

Suitable temperatures for combining the acid with the solution range from about −5° C. to about 35° C., or about 0° C. to about 5° C.

The acid may be added into the reaction mass over an extended period of time, such as from about 0.5 hours to about 10 hours, or about 0.5 hour to about 5 hours. Temperatures for conducting the reaction can range from about −10° C. to about 50° C., or about 0° C. to about 5° C. Increases in temperature can cause a decrease in the purity of the product.

Step c) involves adjusting the pH of the solution of step b) from about 5 to about 7.

The pH of the reaction solution is adjusted to about 5 to about 7 by adding a suitable base.

Suitable bases include, but are not limited to: alkali metal hydroxides such as lithium hydroxide, sodium hydroxide, potassium hydroxide and the like; alkali metal carbonates such as sodium carbonate, potassium carbonate and the like; and alkali metal bicarbonates such as sodium bicarbonate, potassium bicarbonate and the like; as solids or in the form of solutions in water.

Suitable times, over which an aqueous base is added into the solution, range from about 45 minutes to about 3 hours, or about 1 to about 2 hours. Temperatures for adjusting the pH range from about −10° C. to 50° C., or about 0° C. to about 5° C.

Solid simvastatin can be recovered, following step (c). Solid material may be recovered from step (c) with or without cooling below the operating temperature, using any of techniques such as decantation, filtration by gravity or by suction, centrifugation, and the like. The crystals so isolated can carry a small proportion of occluded mother liquor containing a higher percentage of impurities. If desired the crystals can be washed with a substance in which the solid is not appreciably soluble, to wash out the mother liquor.

The obtained solid may be optionally purified by treating its solution with activated charcoal. The obtained solid after step c) is dissolved in a suitable solvent and optionally treated with activated charcoal to enhance the purity of the compound, followed by filtration through a paper, membrane, etc. or a bed of a medium such as flux-calcined diatomaceous earth (Hyflow), to remove the carbon.

The solid product may optionally be further dried. Drying can be suitably carried out using equipment such as a tray dryer, vacuum oven, air oven, fluidized bed drier, spin flash dryer, flash dryer and the like. The drying can be carried out at temperatures of about 40° C. to about 50° C. The drying can be carried out for any desired time periods to achieve the desired product purity, times from about 1 to 20 hours, or longer, frequently being adequate.

In an aspect, the invention includes processes for preparing solid premixes having uniform particle size distributions, an embodiment comprising grinding simvastatin with an antioxidant such as butylated hydroxyanisole, dissolved in a solvent such as methanol.

Simvastatin used in the processes for the preparation of premixes may be either a wet solid cake or a dried solid.

The processes of the present application are simple, improved, eco-friendly, cost-effective, commercially viable, robust and reproducible on an industrial scale.

Having thus described the invention, those in the art will appreciate modifications to the invention as described and illustrated that do not depart from the spirit and scope of the invention as disclosed. The following examples of certain specific aspects and embodiments of the invention are provided solely to assist in understanding various features of the invention but are not intended to, and should not be construed to, limit its scope in any way. The examples do not include detailed descriptions of conventional methods. Such methods are well known to those of ordinary skill in the art and are described in numerous publications.

### EXAMPLE 1

### Preparation of Simvastatin

150 g of simvastatin ammonium salt of Formula II was charged into a round bottom flask containing 300 ml acetonitrile and stirred for about 10-15 minutes, followed by cooling to about 0° C. 150 ml of concentrated hydrochloric acid in 300 ml of water was added slowly through a dropper over about 75 minutes at 3° C. and stirred to completion of the reaction. pH of the solution was adjusted to 6.5 by addition of 600 ml of sodium hydroxide solution (60 g NaOH dissolved in 600 ml water) at 3° C. and stirred for solid formation. The solid was filtered and washed with 300 ml water to afford a 199 g wet cake. The wet cake was charged into a round bottom flask containing 300 ml water, stirred for about 60 minutes at about 28° C., and filtered. The solid was washed with water (150 ml) and suction dried for about 60 minutes to afford 200 g of wet simvastatin. HPLC purity: 98.02%.

### EXAMPLE 2

### Preparation of Simvastatin Premix

The 200 g of wet simvastatin from Example 1 was dissolved in 1200 ml methanol and stirred at 25-30° C. to produce a clear solution. 30 g activated charcoal (SC-40 grade) was added to the solution and stirred for about 60 minutes. The mixture was filtered through cloth and paper and the obtained solution was passed though a 0.45 μm filter paper. The obtained clear solution was charged into a fresh round bottom flask and 0.3 g of butylated hydroxyanisole was added, heated to about 40° C. and circulated through a colloid mill. 2100 ml water was added dropwise over about 1 hour at 35° C., followed by cooling to room temperature and stirring for solid formation. The solid was filtered, washed with 330 ml of water, and suction dried for about one hour.

The obtained wet solid was loaded into a rapid mixture grinder (RMG) and stirred for about 10 minutes. 79 mg of butylated hydroxyanisole dissolved in 10 ml methanol was added uniformly to the solid and stirred under the rapid mixture grinder about 30 minutes. The obtained solid was unloaded from the RMG and dried under vacuum at 45° C. for about 2-3 hours to afford the premix.

### EXAMPLE 3

### Alternate Preparation of Simvastatin

To a suspension of the ammonium salt of a 3,5-dihydroxy acid of Formula II (100 g) in acetonitrile (200 ml), a mixture of 36% hydrochloric acid (100 ml) and water (200 ml) was added at about 0° C. to 5° C., over about 30 to about 45 minutes. The mixture was further stirred at about 0° C. to 5° C. for about 90 minutes. After completion of the reaction, water (200 ml) was charged slowly to the mass and the mixture was stirred for about 20 minutes. The pH of the mass was then adjusted to about 6.2 at about 0° C. to about 5° C. using sodium hydroxide. The mixture was stirred for about 30 minutes followed by filtration and washing the solid with water. The obtained solid was dried to afford 88.5 g of title compound.

### EXAMPLE 4

### Alternate Preparation of Simvastatin Premix

Simvastatin (11.5 Kg) and methanol (138 L) were charged into a reactor and stirred until complete dissolution of the solid. Activated carbon was charged and the mixture was stirred for about 30 minutes. The mass was filtered, washed with methanol (23 L) and subjected to heating to about 38° C. Water (161 L) was added over a period of about 50 minutes and the mass was cooled to a temperature of about 10° C. over a period of 2 hours. The obtained mass was centrifuged and washed with chilled water, and the wet material was spin-dried. Methanol (25.3 L), water (66 L) and butylated hydroxyanisole (0.017 Kg) were charged into a reactor and stirred for about 10 minutes, then the above spin-dried material was charged and the whole mass was stirred for about 30 minutes at about 30° C. The solid obtained was separated by centrifugation and washed with water. The wet material was then spin-dried to afford 10.3 Kg of premix.

We claim:

1. A process for preparing simvastatin, comprising:
a) providing a solution of a 3,5-dihydroxy acid or salt having Formula II, 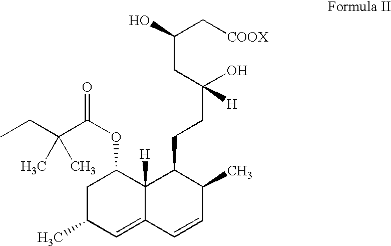
wherein X is H, Na, K, or NH₄, in a solvent;
b) adding an acid in an amount equal or greater than an amount sufficient to protonate the acid or salt; and
c) adjusting pH to about 5 to about 7.

2. The process of claim 1, wherein the solvent is a water miscible organic solvent.

3. The process of claim 1 wherein the solvent comprises acetonitrile.

4. The process of claim 1, wherein acid is added in b) in a single lot.

5. The process of claim 4, wherein acid is added over an extended period of time.

6. The process of claim 1, wherein an acid in b) is an inorganic acid.

7. The process of claim 1, wherein an acid in b) comprises sulphuric acid, hydrochloric acid, or hydrobromic acid.

8. The process of claim 1, wherein an acid in b) comprises hydrochloric acid.

9. The process of claim 1, wherein about 6.5 equivalents of hydrochloric acid is used in b), per equivalent of 3,5-dihydroxy acid or salt.

10. The process of claim 1, wherein a temperature for adding an acid in b) is lower than about 5° C.

11. The process of claim 1, wherein a base in c) comprises an alkali metal hydroxide, carbonate, or bicarbonate.

12. The process of claim 1, wherein a base in c) comprises sodium hydroxide or potassium hydroxide, in solid form or as a solution in water.

13. The process of claim 1, wherein a base in c) comprises a 20% by weight aqueous sodium hydroxide solution.

14. A process for preparing simvastatin, comprising:
a) providing a solution of a 3,5-dihydroxy acid or salt having Formula II, 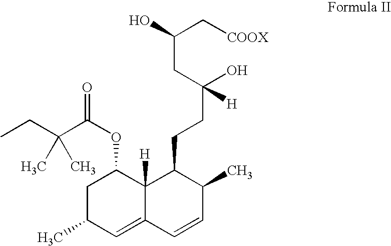
wherein X is H, Na, K, or NH₄, in a solvent comprising acetonitrile;
b) adding an acid in an amount about 4 to about 8 equivalents, per equivalent of 3,5-dihydroxy acid or salt; and
c) adjusting pH to about 5 to about 7.

15. The process of claim 14, wherein a 3,5-dihydroxy acid or salt comprises an ammonium salt.

16. The process of claim 14, wherein an acid in b) comprises hydrochloric acid.

17. The process of claim 14, wherein an acid in b) is added at temperatures less than about 5° C.

18. The process of claim 14, wherein an acid in b) is added in a single lot, over a period of about 0.5 hour to about 5 hours.

19. A process for preparing a solid premix containing simvastatin, comprising grinding simvastatin with a solution comprising an antioxidant.

20. The process of claim 19, wherein an antioxidant comprises butylated hydroxyanisole and a solvent comprises methanol.
